(19) United States
(12) Patent Application Publication
(10) Pub. No.: US 2024/0238164 A1
(43) Pub. Date: Jul. 18, 2024

(54) RETRACTABLE LOW-PROFILE PEDIATRIC GASTROSTOMY TUBE AND USES THEREOF

(71) Applicant: CHILDREN'S NATIONAL MEDICAL CENTER, Washington, DC (US)

(72) Inventors: Mihika AEDLA, Washington, DC (US); Karun SHARMA, Washington, DC (US); Siya ZHANG, Washington, DC (US); Jason QIAN, Washington, DC (US); Charlotte CHENG, Washington, DC (US); Jocelyn HSU, Washington, DC (US); Anson ZHOU, Washington, DC (US); Katherine HU, Washington, DC (US); Kevin SOMPEL, Washington, DC (US)

(73) Assignee: CHILDREN'S NATIONAL MEDICAL CENTER, Washington, DC (US)

(21) Appl. No.: 18/560,257

(22) PCT Filed: May 24, 2022
(86) PCT No.: PCT/US2022/030752

§ 371 Date: Nov. 10, 2023

Related U.S. Application Data
(60) Provisional application No. 63/192,857, filed on May 25, 2021.

(51) Int. Cl. A61J 15/00

(52) U.S. Cl. CPC A61J 15/0015; A61J 15/0026

(57) ABSTRACT

A gastrostomy tube for insertion through an abdomen and into a stomach, including a cylindrical outer tube including a first proximal opening and a first distal opening, a flange surrounding the first proximal opening of the outer tube, a detachable cylindrical inner tube including a second proximal opening and a second distal opening, and one or more spokes attached to a distal end of the gastrostomy tube, wherein the one or more spokes include an actuatable portion configured to rotate the one or more spokes between a retracted position and an expanded position, wherein the inner tube is insertable into the first proximal opening of the outer tube, wherein the one or more spokes are rotatable into the expanded position when the inner tube is inserted through the first distal opening of the outer tube, and wherein the one or more spokes are substantially perpendicular to the outer tube in the expanded position.

### CROSS-REFERENCE TO RELATED APPLICATIONS

The present application claims priority to U.S. Provisional Application No. 63/192,857, filed May 25, 2021, the entire content of each of which is incorporated by reference herein in its entirety for all purposes.

### BACKGROUND

### Field of the Disclosure

The present disclosure relates to gastrostomy tubes.

### Description of the Related Art

Gastrostomy tubes are inserted into the stomach of a patient in order to directly provide food and/or medicine to the stomach. Gastrostomy tubes are initially inserted via a surgical procedure wherein an incision is made through the abdomen and into the stomach to create an opening for placement of the gastrostomy tube. Feeding tubes can be attached as extensions to gastrostomy tubes to connect a food source to the stomach. As a long-term solution, gastrostomy tubes must be stable and should allow feeding tubes to be inserted and removed easily after a patient is discharged from a hospital setting. Gastrostomy tube placement is a common surgical procedure in the pediatric population, as 1.4 million children in America may require feeding assistance each year.

The foregoing “Background” description is for the purpose of generally presenting the context of the disclosure. Work of the inventors, to the extent it is described in this background section, as well as aspects of the description which may not otherwise qualify as prior art at the time of filing, are neither expressly or impliedly admitted as prior art against the present disclosure.

### SUMMARY

In one embodiment, the present disclosure is related to a gastrostomy tube for insertion through an abdomen and into a stomach, including a cylindrical outer tube including a proximal opening and a distal opening, a flange surrounding the proximal opening of the outer tube, a detachable cylindrical inner tube including a proximal opening and a distal opening, and one or more spokes attached to a distal end of the gastrostomy tube, wherein the one or more spokes include an actuatable portion configured to rotate the one or more spokes between a retracted position and an expanded position, wherein the inner tube is insertable into the proximal opening of the outer tube, wherein the one or more spokes are rotatable into the expanded position when the inner tube is inserted through the distal opening of the outer tube, and wherein the one or more spokes are substantially perpendicular to the outer tube in the expanded position.

In one embodiment, the present disclosure is related to a method for inserting a gastrostomy tube through an abdomen and into a stomach, the method including arranging one or more spokes attached to a distal opening of the gastrostomy tube in a retracted position, inserting an outer tube of the gastrostomy tube through the abdomen and into the stomach, inserting an inner tube into the outer tube, engaging, with the inner tube, an actuatable portion of the one or more spokes to rotate the one or more spokes into an expanded position, the one or more spokes being substantially perpendicular to the outer tube in the expanded position, and locking the inner tube inside the outer tube.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is an illustration of a gastrostomy tube according to one embodiment;

FIG. 1B is an illustration of a gastrostomy tube according to one embodiment;

FIG. 1C is an illustration of an outer tube of a gastrostomy tube according to one embodiment;

FIG. 1D is an illustration of an inner tube of a gastrostomy tube according to one embodiment;

FIG. 2A is an illustration of a placement of a gastrostomy tube according to one embodiment;

FIG. 2B is an illustration of a position of a gastrostomy tube according to one embodiment;

FIG. 2C is an illustration of a placement of an inner tube inside an outer tube according to one embodiment;

FIG. 3A is an illustration of an extension tube attached to an inner tube according to one embodiment;

FIG. 3B is an illustration of an extension tube, an inner tube, and an outer tube according to one embodiment;

FIG. 3C is an illustration of an extension tube, an inner tube, and an outer tube according to one embodiment;

FIG. 3D is an illustration of an extension tube attached to an inner tube and an inner tube attached to an outer tube according to one embodiment;

FIG. 4A is an illustration of a spoke according to one embodiment;

FIG. 4B is an illustration of a petal-shaped spoke according to one embodiment;

FIG. 4C is an illustration of an inner spoke and a sheath according to one embodiment;

FIG. 5A is an illustration of a spoke and a flexible joint in a retracted position according to one embodiment;

FIG. 5B is an illustration of a spoke and a flexible joint in a retracted position according to one embodiment;

FIG. 5C is an illustration of an inner spoke and a sheath attached to a flexible joint according to one embodiment;

FIG. 5D is an illustration of a spoke and a flexible joint in an expanded position according to one embodiment;

FIG. 6A is an illustration of a gastrostomy tube with spokes attached with flexible joints in a retracted position according to one embodiment;

FIG. 6B is an illustration of a gastrostomy tube with spokes attached with flexible joints in a retracted position according to one embodiment;

FIG. 6C is an illustration of a gastrostomy tube with spokes attached with flexible joints in a retracted position according to one embodiment;

FIG. 7A is an illustration of a gastrostomy tube with spokes attached with flexible joints in an expanded position according to one embodiment;

FIG. 7B is an illustration of a gastrostomy tube with spokes attached with flexible joints in an expanded position according to one embodiment;

FIG. 7C is an illustration of a gastrostomy tube with spokes attached with flexible joints in an expanded position according to one embodiment;

FIG. 8A is an illustration of an inner tube with flexible spokes according to one embodiment;

FIG. 8B is an illustration of an inner tube with flexible spokes according to one embodiment;

FIG. 9A is an illustration of a gastrostomy tube with flexible spokes in a retracted position according to one embodiment;

FIG. 9B is an illustration of a gastrostomy tube with flexible spokes in a retracted position according to one embodiment;

FIG. 10A is an illustration of a gastrostomy tube with flexible spokes in an expanded position according to one embodiment:

FIG. 10B is an illustration of a gastrostomy tube with flexible spokes in an expanded position according to one embodiment;

FIG. 11 is an illustration of a spoke with a hinge attachment to an outer tube according to one embodiment;

FIG. 12A is an illustration of an outer tube with cutouts for a hinge attachment according to one embodiment;

FIG. 12B is an illustration of an outer tube with cutouts for a hinge attachment according to one embodiment;

FIG. 13A is an illustration of a gastrostomy tube with hinged spokes in a retracted position according to one embodiment;

FIG. 13B is an illustration of a gastrostomy tube with hinged spokes in a retracted position according to one embodiment;

FIG. 14A is an illustration of a gastrostomy tube with hinged spokes in an expanded position according to one embodiment;

FIG. 14B is an illustration of a gastrostomy tube with hinged spokes in an expanded position according to one embodiment;

FIG. 15A is an illustration of a spoke attached to an outer tube with an axle according to one embodiment;

FIG. 15B is an illustration of a spoke with an axle according to one embodiment:

FIG. 16A is an illustration of an outer tube with cutouts for an axle attachment according to one embodiment;

FIG. 16B is an illustration of an outer tube with cutouts for an axle attachment according to one embodiment;

FIG. 17A is an illustration of a gastrostomy tube with spokes attached with axles in a retracted position according to one embodiment;

FIG. 17B is an illustration of a gastrostomy tube with spokes attached with axles in a retracted position according to one embodiment;

FIG. 18A is an illustration of a gastrostomy tube with spokes attached with axles in an expanded position according to one embodiment;

FIG. 18B is an illustration of a gastrostomy tube with spokes attached with axles in an expanded position according to one embodiment;

FIG. 19A is an illustration of a gastrostomy tube with a membrane extending from spokes attached to an outer tube according to one embodiment;

FIG. 19B is an illustration of a gastrostomy tube with a membrane extending from spokes attached to an inner tube according to one embodiment;

FIG. 20 is an illustration of a gastrostomy tube with a biodegradable sheath according to one embodiment; and

FIG. 21 illustrates a method for using the gastrostomy tube according to one embodiment.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Gastrostomy tubes can be anchored to an opening in the stomach or to the inside of the gastric wall to be used over extended periods of time. Typical gastrostomy tubes use physical mechanisms such as balloons or capsules to secure the tube in the stomach. However, current approaches for anchoring gastrostomy tubes are not user-friendly and carry risks of malfunction. For example, balloons are prone to deflation, which results in the tube becoming unsealed and dislodging from the gastric wall. Re-inflating balloons can be complicated and pose risks, especially when performed by untrained professionals outside of a hospital setting. Capsules require a large amount of force to deploy and to attach extension tubing, which can cause pain for patients. Unwanted movement of gastrostomy tubes in the stomach can result in infection, pain, internal or external injury, and/or inability to properly feed a patient. Gastrostomy tube failures can result in hospital readmission in order to fix displacement and/or replace tubes. A novel design of a more stable gastrostomy tubes can reduce hospital readmission rates by 14% and save patients and their families a S47M cost burden.

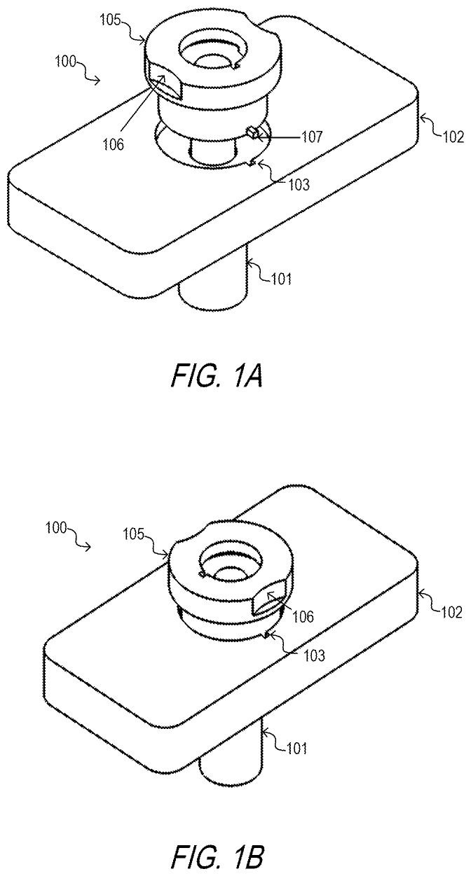

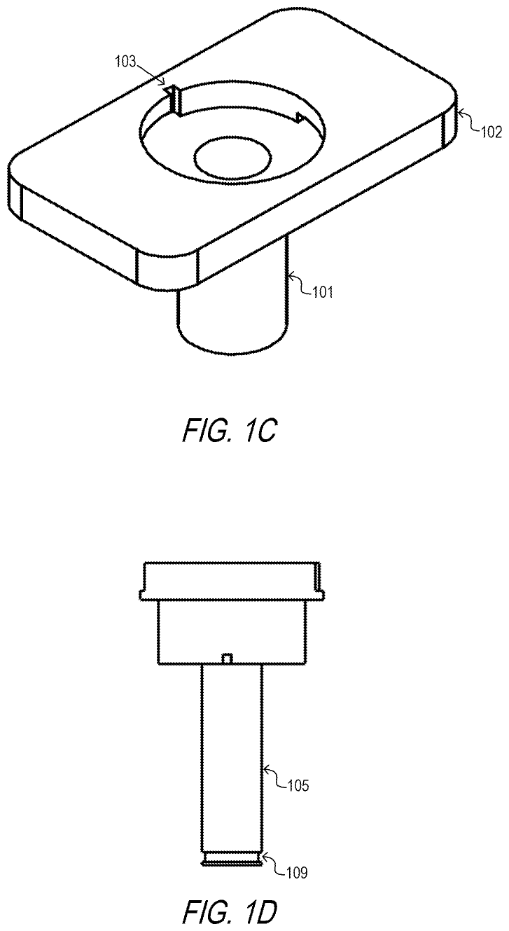

In one embodiment, the present disclosure is related to a gastrostomy tube for secure insertion into a patient's abdomen and stomach. In one embodiment, the gastrostomy tube can include a hollow, cylindrical outer tube and a hollow, cylindrical inner tube wherein a circumference of the outer tube is larger than a circumference of the inner tube. The gastrostomy tube can be handled at the proximal end, while the distal end of the gastrostomy tube can be inserted into the stomach. The outer tube and the inner tube can be hollow and open on both ends. The outer tube can be inserted through the abdomen and into the stomach. The inner tube can be inserted concentrically into the outer tube to engage a mechanism that fixes the outer tube to the gastric wall. In one embodiment, the inner tube can provide access to a feeding tube. FIG. 1A illustrates an example embodiment of a gastrostomy tube 100 with an outer tube 101 and an inner tube 105. The outer tube 101 can be surrounded by a flange 102 at the proximal end, wherein the flange 102 can rest on top of the abdomen when the outer tube 101 is inserted. The flange 102 can be wider than the incision in the abdomen in order to remain external to the patient's body when the outer tube is inserted. In one embodiment, the flange can be composed of a foam or foamlike material to minimize pressure and friction when the flange is in contact with the skin. In another embodiment, the bottom surface of the flange can be a foam or foamlike material, while the rest of the flange can be made of an alternative material. In one embodiment, the flange can be padded, e.g., along the bottom surface of the flange. In one embodiment, the flange can be coated or infused with an antimicrobial or antiseptic agent to minimize infection, especially at the incision in the abdomen. In one embodiment, the inner tube 105 can include finger holds 106 at a proximal end of the inner tube for gripping and inserting the inner tube 105.

In one embodiment, the proximal end of the inner tube 105 can protrude above the top of the flange 102 when the inner tube 105 is fully inserted into the outer tube 101. In one embodiment, the inner tube can be locked into the outer tube. In one embodiment, the locking mechanism can be an industry-standard locking mechanism for medical tubing. For example, the locking mechanism can be a Luer lock. In another embodiment, the locking mechanism can be located along the body of the inner tube and/or the outer tube. In one embodiment, the inner tube 105 can include a locking tab 107 at the proximal end. FIG. 1B illustrates an example embodiment of the gastrostomy tube 100 when the inner tube 105 is inserted into the outer tube 101. The locking tab can be inserted into a notch 103 in the flange 102 of the outer tube 101. FIG. 1C illustrates an example embodiment of the outer tube 101 of the gastrostomy tube 100. The notch 103 in the flange 102 can be connected to a groove along the inner wall of the flange 102. In one embodiment, the inner tube 105 cannot be inserted into or removed from the outer tube 101 unless the locking tab is aligned with the notch 103 in the flange 102. The inner tube 105 can be rotated so that the locking tab runs along the groove along the inner wall of the flange 102 and is no longer aligned with the notch 103, thus locking the inner tube 105 in the outer tube 101. The inner tube 105 must then be rotated again to align the locking tab with the notch and remove the inner tube. Locking the inner tube 105 inside the outer tube 101 can prevent the patient or others from accidentally removing the inner tube 105. In one embodiment, the flange 102 can include a plurality of parallel grooves at varying depths along the inner wall of the flange. The plurality of grooves can enable the inner tube 105 to be locked at varying degrees of insertion into the outer tube 101.

In one embodiment, the gastrostomy tube can include circumferential notches or ridges to provide discrete steps or levels for inserting the inner tube 105 into the outer tube 101. For example, the inner wall of the outer tube 101 can include at least one notch or ridge provide discrete steps or levels for inserting the inner tube 105 into the outer tube 101. As another example, the inner tube 105 can include at least one notch or ridge along the outer wall of the inner tube 105. FIG. 1D illustrates an example of an inner tube 105 with a notch 109 near the distal opening. The notch 109 can provide tactile resistance to a user indicating how far the inner tube 105 has been inserted into the outer tube. In one embodiment, the notches can correspond to lengths of the inner tube corresponding to feeding functionalities and/or to patient anatomy.

In one embodiment, the gastrostomy tube can include a safety mechanism to prevent unintentional turning and removal of the inner tube. As an example embodiment, the safety mechanism can require a squeezing force to be applied to the inner tube or the outer tube in order to rotate the inner tube. For example, the inner tube and/or the outer tube can include tabs that must be pressed simultaneously in order to unlock and remove the inner tube. In one embodiment, the safety mechanism can be an industry-standard safety mechanism for securing two components.

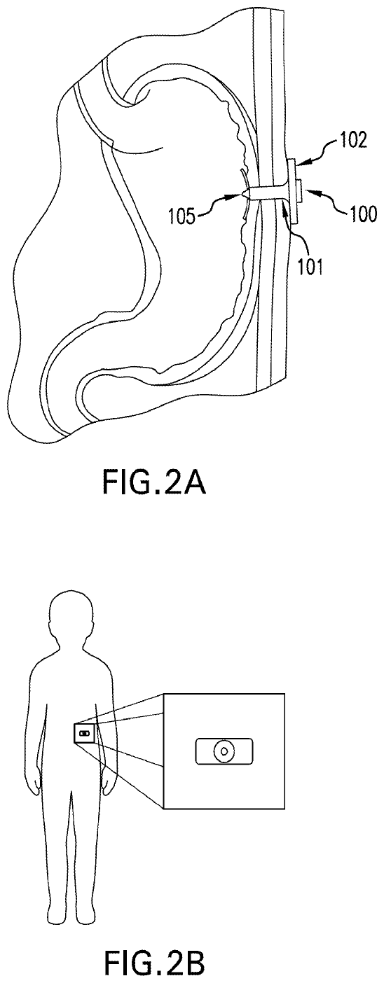

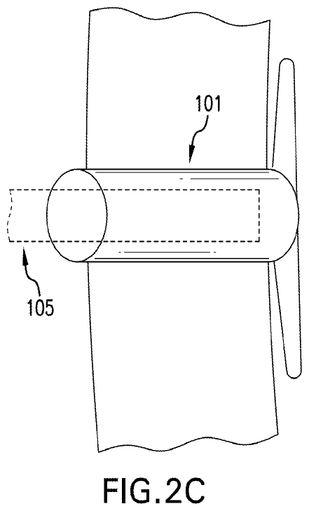

FIG. 2A illustrates an example of a gastrostomy tube 100 inserted into the stomach through the abdomen. The flange 102 of the outer tube 101 can sit against the abdomen. The outer tube 101 and the inner tube 105 can extend through the abdominal wall and the gastric wall to reach the interior of the stomach. The outer tube 101 can be fixed to the interior of the gastric wall. FIG. 2B illustrates an example of where the gastrostomy tube 100 can be inserted into the body at the abdomen. FIG. 2C illustrates an example of how the inner tube 105 can be inserted into the outer tube 101 once the outer tube 101 has been inserted into the stomach. In one embodiment, the inner tube 105 can extend past the distal opening of the outer tube 101. In another embodiment, the distal opening of the inner tube 105 can be aligned with the distal opening of the outer tube 101. In one embodiment, the inner tube 105 can extend past the stomach and further down the gastrointestinal tract. In one embodiment, the inner tube 105 can be flexible and/or curved. Advantageously, the inner tube can be inserted into and removed from the outer tube while the outer tube can remain in the stomach. For example, the inner tube can be removed in case of clogging or other complication. The inner tube can be easily replaced with another inner tube.

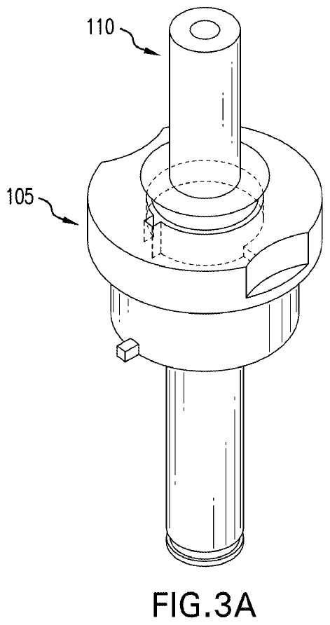

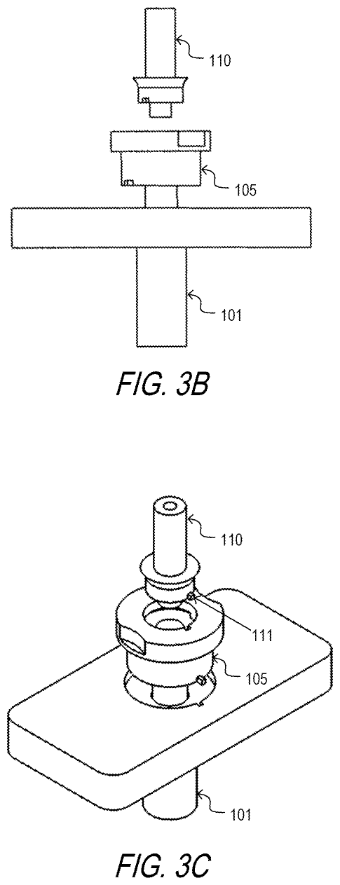

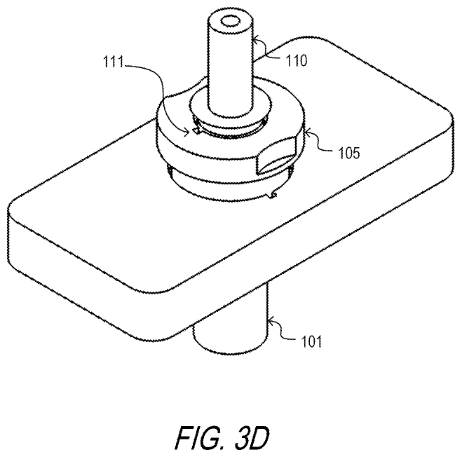

FIG. 3A illustrates an example of a feeding tube 110 inserted into the inner tube 105. The feeding tube 110 can be connected to a feeding bag or other apparatus to provide food directly to the stomach through the gastrostomy tube. In one embodiment, the feeding tube and the gastrostomy tube can be used with a feeding pump. FIG. 3B illustrates an exemplary side view of the feeding tube 110, the inner tube 105, and the outer tube 101. FIG. 3C illustrates an example embodiment of the feeding tube 110 inserted into the inner tube 105 and the inner tube 105 inserted into the outer tube 101. In one embodiment, the feeding tube 110 can be securely fastened to the inner tube 105 using a locking mechanism, e.g., a Luer lock, a locking tab. In one embodiment, the feeding tube 110 can include a locking tab 111 for locking the feeding tube 110 into a groove along the inner wall of the inner tube 105. The feeding tube 110 can fit inside the inner tube 105 as illustrated in FIG. 3D and be securely fastened to the opening in the inner tube 105. The inner tube 105 can similarly fit inside the outer tube 101 and be securely fastened to the outer tube 101. In one embodiment, the feeding tube 110 can be inserted through the inner tube 105 and directly into the stomach. In one embodiment, the body of the feeding tube can be fastened to the inner tube 105. In another embodiment, food or other materials can be introduced to the stomach directly through the inner tube. In one embodiment, a cap can be placed on the proximal opening of the inner tube when a feeding tube is not attached to the inner tube. The cap can be made of silicone. In one embodiment, the cap can be a plug inserted into proximal opening of the inner tube in place of a feeding tube. In one embodiment, the cap or plug can be fastened using an industry-standard fastener design, e.g., a clip-on cap, an adhesive, a magnetic fastener, a screw-on cap. The cap or plug can ensure that unwanted materials do not enter the stomach and that the inner tube remains unobstructed when the patient is not being fed. In one embodiment, a cap or plug can be inserted into the proximal opening of the outer tube when the inner tube is removed from the outer tube.

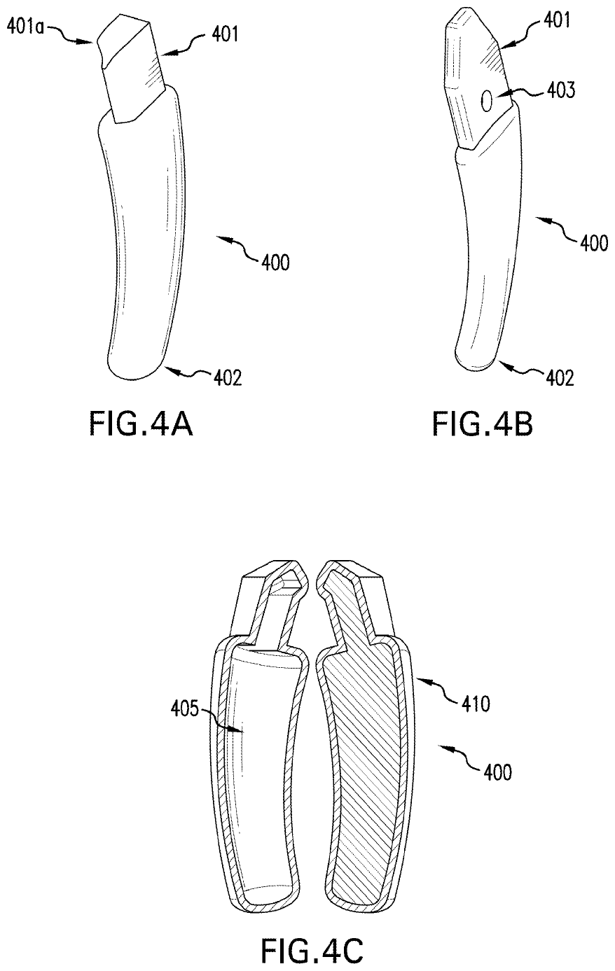

The outer tube can be affixed to the inside of the gastric wall so that the gastrostomy tube does not become dislodged from the opening in the abdomen and stomach. Movement of the gastrostomy tube can cause injury to the stomach and discomfort and can also prevent proper use of the gastrostomy tube for feeding. In one embodiment, the outer tube can be fixed to stomach using at least one spoke attached to the distal end of the gastrostomy tube. The at least one spoke can be attached to the gastrostomy tube and sit along the inside of the gastric wall, as illustrated in FIG. 2A. Example embodiments of spokes are illustrated in FIG. 4A through FIG. 4C.

In one embodiment, the spoke can be curved, as illustrated in FIG. 4A. According to one embodiment, the spoke 400 can be convex, and the radius of the curve of the spoke can be approximately equal to the inner radius of the greater curve of the stomach where the gastrostomy tube is inserted. The curvature of the spoke can vary. A proximal end 401 of the spoke can be attached to the outer tube or the inner tube of the gastrostomy tube. In one embodiment, the proximal end 401 of the spoke can include a tab 401a. In one embodiment, the tab 401a can be a flat surface extending perpendicular to the spoke. The tab 401 can extend past the inner surface of the spoke. In one embodiment, the tab 401a can be tapered. In another embodiment, the tab 401a can be consistent in thickness and shape. In one embodiment, the tab 401a can be straight rather than curved. Alternative thicknesses and dimensions of the tab 401a are also compatible with the present disclosure. In one embodiment, the spoke 400 can have a tapered distal end 402, as illustrated in FIG. 4A. In another embodiment, the spoke 400 can have a petal shape with a wider midsection leading to a tapered distal end 402, as illustrated in FIG. 4B. In one embodiment, the spoke 400 can include at least one opening 403 for the insertion of strips, pins, and/or hinges to affix the proximal end 401 of the spoke 400 to the gastrostomy tube, as will be described in further detail below. The at least one opening can include, but is not limited to, pockets, slits, channels, and/or holes.

In one embodiment, the spoke can be made of a biocompatible material, e.g., medical-grade silicone. In one embodiment, the spoke can be composed of a variety of materials. For example, the distal end of the spoke can be a stiffer material than the body of the spoke. Examples of alternative materials for the spoke 400 can include, but are not limited to, plastics or metals. In one embodiment, the spoke 400 can include an inner spoke 405 encased in a sheath 410, as illustrated in FIG. 4C. The inner spoke can be composed of a stiffer material, and the sheath can be composed of a softer material to prevent irritation of the gastric wall. In one embodiment, the surface of the inner spoke 405 can include ridges or other modulations to create friction between the surface of the inner spoke 405 and the inner surface of the sheath 410. The modulations can improve the grip of the sheath 410 on the inner spoke 405. Additionally or alternatively, the modulations can be on the inner surface of the sheath 410.

The proximal end of the spokes can be attached to the gastrostomy tube. In an exemplary embodiment, four spokes can be attached to the gastrostomy tube to secure the tube to the stomach. The four spokes can be equally spaced around the distal end of the gastrostomy tube. Alternative numbers of spokes and positioning of spokes around the gastrostomy tube are also compatible with the present disclosure. In one embodiment, the spokes can be attached along the body of the gastrostomy tube. In one embodiment, a mechanism can be used to engage the spoke in a retracted position or an expanded position. In the retracted position, the spoke can be approximately vertical and point downwards along the longitudinal axis of the gastrostomy tube. In one embodiment, the distal ends of the spokes can meet at an endpoint when the spokes are in a retracted position. In another embodiment, the distal ends of the spokes do not touch in the retracted position. In the expanded position, the spoke can flare outwards from the distal opening of the gastrostomy tube. The spokes can be fixed in a wide range of angles and positions in the expanded position. In a preferred embodiment, the upper surfaces of the spokes can be in contact with the inner gastric wall in the expanded position. The spokes can then fix the outer tube against the stomach so that the outer tube cannot be removed from the stomach when the spokes are in the expanded position. In one embodiment, the maximum angle of the spokes in the expanded position can be approximately perpendicular to the longitudinal axis of the outer tube. The maximum angle can ensure that the spokes do not point upwards, e.g., towards the flange, as this configuration could irritate the inside of the gastric wall and/or cause pain to the patient.

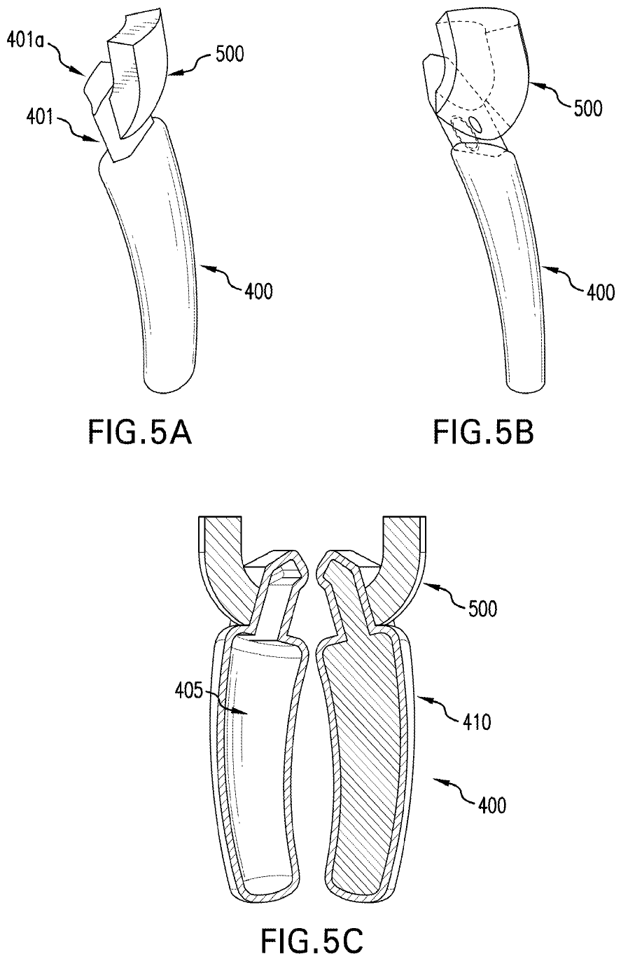

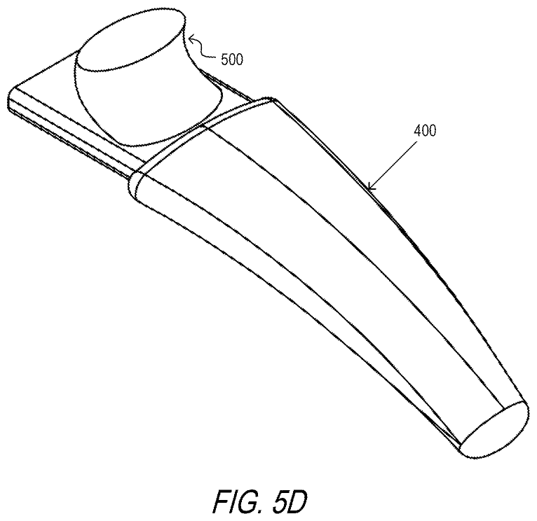

FIG. 5A through FIG. 5D illustrate a flexible joint 500 used to attach the spoke 400 to the gastrostomy tube. The flexible joint 500 can be attached to the spoke 400 near the proximal end 401 of the spoke 400. The flexible joint can bend from a convex shape to a concave shape. FIG. 5A illustrates the flexible joint 500 in a convex shape according to an exemplary embodiment of the gastrostomy tube. The spoke 400 can be maintained in a retracted position when the flexible joint 500 is in the convex shape. FIG. 5B illustrates another view of the flexible joint 500 in a convex shape attached to the spoke 400 according to an exemplary embodiment of the gastrostomy tube. In one embodiment, the flexible joint 500 can fit into a divot or opening in the proximal end of the spoke 400. In one embodiment, the flexible joint 500 can be attached to the sheath 410 surrounding an inner spoke 405, as illustrated in FIG. 5C. FIG. 5D illustrates the flexible joint 500 in a concave shape according to an exemplary embodiment of the gastrostomy tube. The concave shape of the flexible joint 500 can engage the spoke in an expanded state. The point of contact between the flexible joint 500 and the spoke 400 can remain fixed regardless of whether the flexible joint 500 is in the convex shape or the concave shape. Likewise, the point of contact between the flexible joint 500 and the gastrostomy tube can remain fixed regardless of whether the flexible joint 500 is in the convex shape or the concave shape.

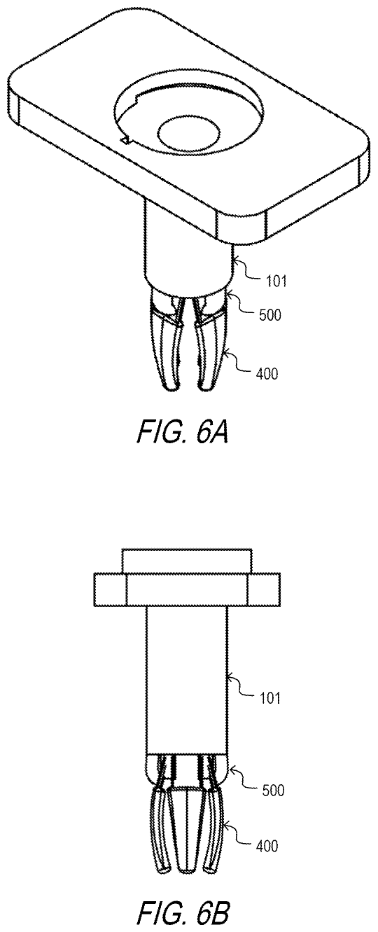

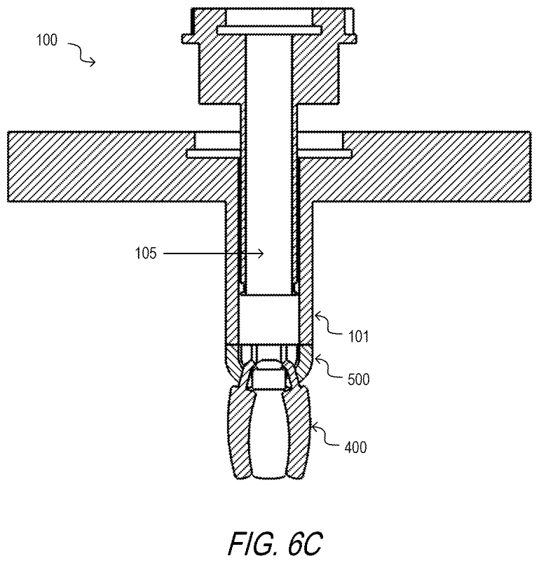

The flexible joint can be used to attach the spoke to the outer tube of the gastrostomy tube. FIG. 6A and FIG. 6B illustrate spokes 400 attached to the outer tube 101 via flexible joints 500 according to an exemplary embodiment of the gastrostomy tube, wherein the spokes 400 are in a retracted position. In one embodiment, the outer tube 101, the flexible joint 500, and the spokes 400 can be a single, contiguous piece. In another embodiment, the flexible joint 500 can be attached to the outer tube 101, e.g., via a biocompatible adhesive. The spokes can point downwards when the flexible joint 500 is in a convex shape. The retracted position can create a streamlined shape that enables the outer tube to be inserted easily into the abdomen and the stomach. FIG. 6C is a transparent illustration of the gastrostomy tube 100 with spokes 400 attached via flexible joints 500 in a retracted position, according to one embodiment. The inner tube 105 is not completely inserted into the outer tube 101. The distal end of the inner tube 105 sits above or against the top of the spokes 400.

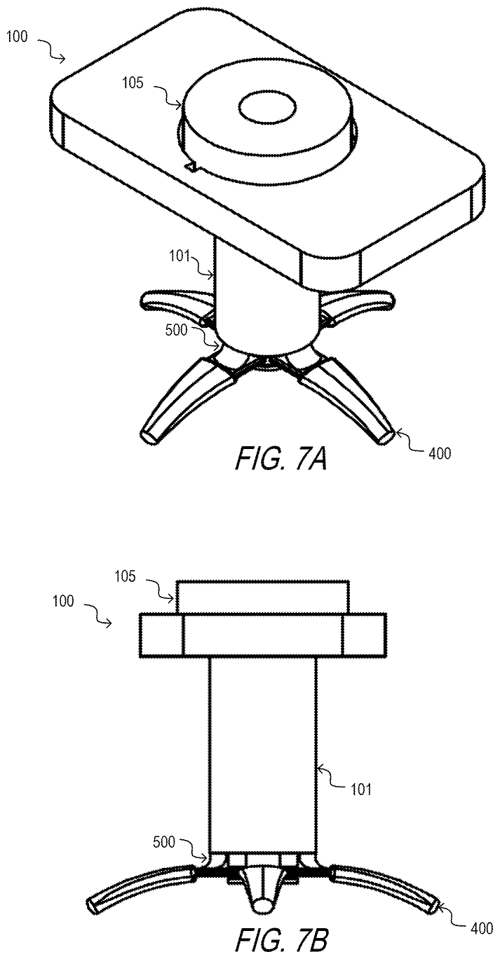

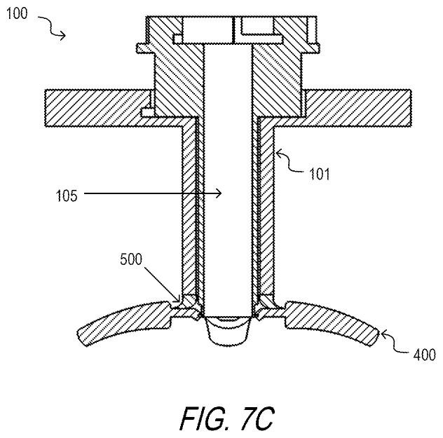

FIG. 7A and FIG. 7B illustrate spokes 400 attached to the outer tube 101 via flexible joints 500 in an expanded position according to an exemplary embodiment of the gastrostomy tube. The flexible joints 500 are bent in a concave shape, enabling the spokes 400 to rotate outwards from the distal end of the outer tube 101. The spokes 400 can be positioned against the inside of the gastric wall when in the expanded position in order to hold the gastrostomy tube in place. When the spokes 400 are in the expanded position, the outer tube 101 cannot be pulled out of the stomach because the flared spokes are wider than the opening in the stomach through which the gastrostomy tube has been inserted. In one embodiment, the inner tube 105 can be used to engage the flexible joints and rotate the spokes 400 into the expanded position. When the inner tube 105 is inserted into the outer tube 101, the inner tube 105 can push against the tab at the proximal end of the spoke above the flexible joint 500. The proximal ends of the spokes are fixed to the outer tube 101 by the flexible joints 500 and cannot be displaced downward. Therefore, the downward force applied by the inner tube 105 to the tab can cause the flexible joints 500 to bend from a convex shape to a concave shape, resulting in the spokes 400 being rotated into the expanded position. In one embodiment, the flexible joints 500 can have a continuous range of motion between a convex shape and a concave shape. Thus, the spokes 400 can be fixed in a wide range of positions and angles relative to the gastrostomy tube in the expanded position. In one embodiment, the angle of the spokes 400 in the expanded position can be dependent on how far the inner tube 105 is inserted into the outer tube 101. Inserting the inner tube 105 further into the outer tube 101 can result in the spokes 400 rotating more, e.g., forming a right angle with the longitudinal axis of the gastrostomy tube. In one embodiment, the angle of the spokes 400 in the expanded position can depend on the curvature of the gastric wall. For example, the inner tube 105 can be inserted until the spokes 400 expand to meet the inside of the gastric wall.

FIG. 7C is a transparent illustration of the gastrostomy tube 100 in an expanded position, according to one embodiment. The inner tube 105 is inserted into the outer tube, and the distal end of the inner tube 105 applies a downward force to the spokes 400. The flexible joints 500 are pushed to a concave shape, enabling the spokes 400 to flare out in the expanded position. In one embodiment, the inner tube 105 can extend past the opening of the outer tube 101 into the stomach, as illustrated in FIG. 7C. In another embodiment, the distal end of the inner tube 105 can sit against the proximal end of the spokes 400. In one embodiment, the flexible joints can return to a convex shape when the force on the tabs is removed. Removing the inner tube 105 can cause the flexible joints 500 to bend from the concave shape to the convex shape and the spokes to rotate from the expanded position to the retracted position. Removing the inner tube 105 can prepare the outer tube 101 to be removed from the stomach.

The use of flexible joints can be advantageous in reducing the number of components and empty spaces in the gastrostomy tube. Human tissue can grow around foreign objects that are inserted into the body. Small gaps and components in the foreign objects can provide spaces and/or surfaces for tissue to grow in. The tissue growth can cause complications in removing the foreign objects. In addition, biological materials and/or food particles can be trapped in small spaces. The flexible joints can attach the spokes to the outer body of the gastrostomy tube while minimizing potential surfaces for unwanted tissue growth. The flexible joints can also provide a wide, continuous range of motion for the spokes.

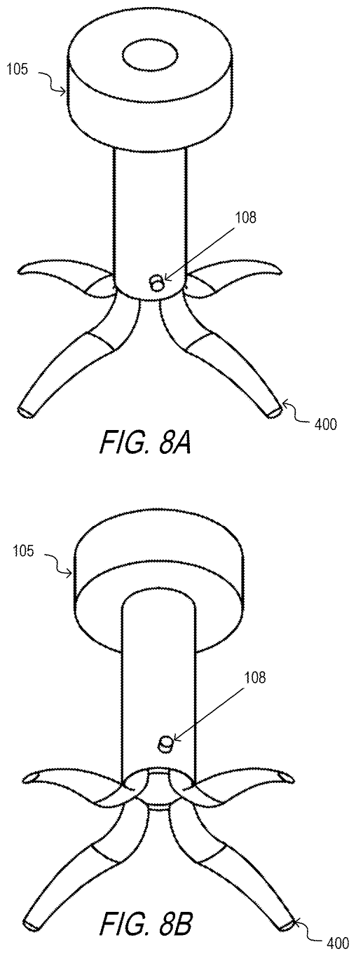

In one embodiment, the spokes can be attached to the inner tube rather than the outer tube and can be made of a flexible material, as illustrated in FIG. 8A and FIG. 8B. In the example embodiment of FIG. 8A, there can be four spokes 400 spaced equally around the distal end of the inner tube 105. In one embodiment, the spokes can be flexible throughout the length of the spokes or at least a region of the spokes. For example, an upper or proximal region of each spoke can be more flexible than a lower or distal region of the spoke. The upper region can be, for example, in the top half of the spoke. In another example embodiment, the upper region can be a top third of the spoke. In one embodiment, the flexible region can be a joint in the spoke. In one embodiment, the resting or natural position of the flexible spokes 400 can be the expanded position, as depicted in FIG. 8A and FIG. 8B. In one embodiment, the body of the inner tube 105 can include a locking tab 108. The locking tab on the body of the inner tube can be in addition to or in place of the locking tab 107 on the top of the inner tube. The locking tab 108 on the body of the inner tube 105 can be used to insert and secure the inner tube inside the outer tube. In one embodiment, the spokes and the inner tube can be made as a single component. In one embodiment, the spokes can be attached to the inner wall of the inner tube. In another embodiment, the spokes can be attached to the circumference of the distal opening of the inner tube.

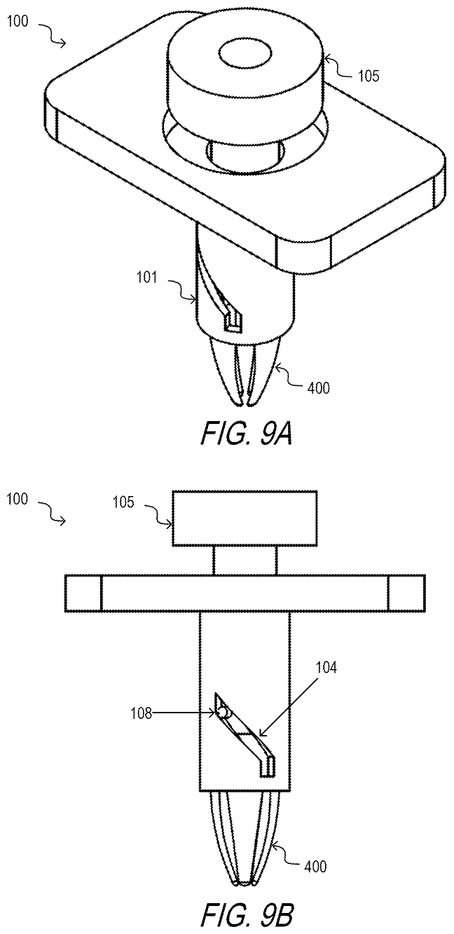

FIG. 9A illustrates a gastrostomy tube 100 with flexible spokes 400 in a retracted position. A force can be applied to bend the flexible spokes 400 into a retracted position. In one embodiment, the distal ends of the spokes can meet at a single point in the retracted position. The spokes 400 and the inner tube 105 can then be inserted into the outer tube 101. In one embodiment, the spoke can be made of a uniform flexible material. The flexible material can be flexible enough to bend due to pressure from the surrounding outer tube. The flexible spokes 400 can be maintained in the retracted position when the inner tube 105 is inserted or partially inserted into the outer tube 101. The outer tube 101 can constrain the flexible spokes 400 to prevent the spokes from bending to an expanded position.

FIG. 9B illustrates a profile view of a gastrostomy tube 100 with flexible spokes 400 in a retracted position according to one embodiment. In one embodiment, the locking tab 108 on the body of the inner tube 105 can fit into a groove 104 in the body of the outer tube 101. The groove 104 can be a designated path that the locking tab 108 must fit into in order for the inner tube 105 to be pushed further into the outer tube 101. In one embodiment, the groove 104 can extend diagonally across the body of the outer tube 101, as depicted in FIG. 9B. The inner tube 105 can be rotated as it is pushed into the outer tube 101 in order to fit the locking tab 108 into the groove 104. The shape of the groove 104 can allow the inner tube 105 to be locked into the outer tube 101. The inner tube 105 cannot be removed from the outer tube 101 with only a pulling force. The inner tube 105 must be rotated and pulled at the same time in order for the locking tab 108 to travel up through the groove 104 to remove the inner tube 105 from the outer tube 101. The locking mechanism can secure the inner tube 105 and prevent the inner tube from falling out of the outer tube or being accidentally removed.

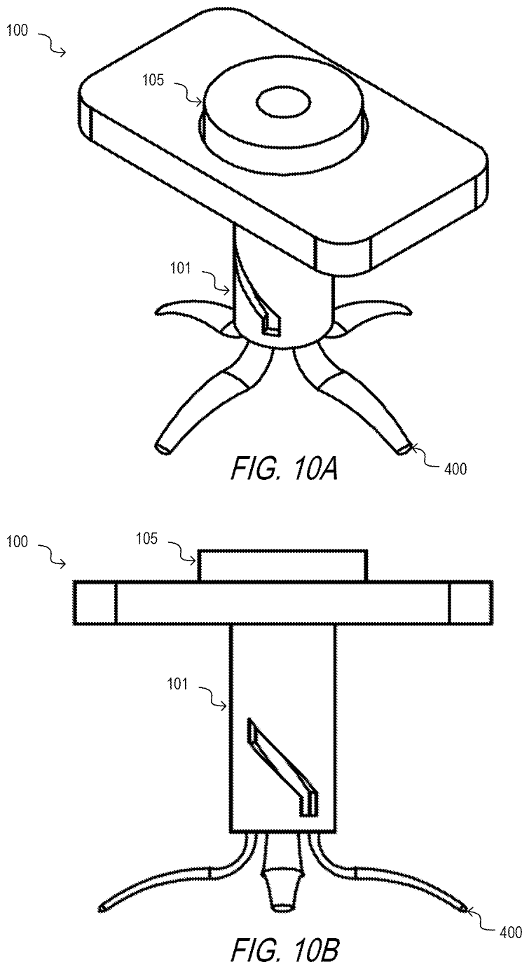

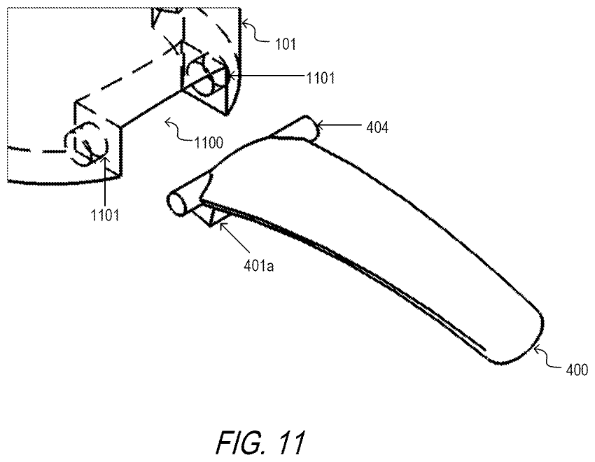

FIG. 10A illustrates an example embodiment of a gastrostomy tube 100 with flexible spokes 400 in an expanded position. In the expanded position, the inner tube 105 is inserted into the outer tube 101 until the flexible spokes 400 are pushed through the distal opening of the outer tube 101. The flexible spokes 400 can then bend into the expanded position without being constricted by the outer tube. In one embodiment, the flexible spokes 400 can bend even if a portion of the spokes are still covered by the outer tube 101. In one embodiment, the flexible spoke 400 can bend more as more of the spoke emerges from the distal opening of the outer tube. Additional force is not required to engage the flexible spoke 400 in the expanded position once the spoke has been pushed through the distal opening of the outer tube. FIG. 10B is a profile illustration of a gastrostomy tube 100 with flexible spokes 400 in the expanded position. In one embodiment, the inner tube 105 can emerge from the distal opening of the outer tube 101.

In one embodiment, removing the inner tube 105 can result in the flexible spokes 400 returning to a retracted position. Pulling the inner tube 105 up through the outer tube 101 can cause the flexible spokes 400 to bend into the retracted position to fit into the distal opening of the outer tube. The retracted position can be maintained while the flexible spokes 400 are inside of the outer tube 101. In one embodiment, the flexible spokes 400 can return to the expanded position when the inner tube is fully removed from the outer tube 101. In one embodiment, the use of flexible spokes 400 can also reduce the number of components and/or empty space in the gastrostomy tube. In one embodiment, the flexible spokes 400 and the inner tube 105 can be formed as a single component. In another embodiment, the flexible spokes 400 can be attached to the inner tube 105, e.g., using a biocompatible adhesive.

In one embodiment, the spokes can be attached to the outer body of the gastrostomy tube using a hinge. The spokes can be semi-flexible or rigid. FIG. 11 illustrates an example embodiment of a hinged spoke attaching to the outer tube of a gastrostomy tube. The outer tube 101 can include at least one cutout 1100 at the distal opening. The spoke 400 can be inserted into the cutout. In one embodiment, the cutout 1100 can be approximately rectangular. In another embodiment, the cutout 1100 can be rounded. The width of the cutout 1100 can be approximately the width of the body of the spoke. The cutout 1100 can include a hole 1101 on either side of the cutout 1100. The spoke 400 can include a rod 404 at the proximal end of the spoke 400. The rod can be rigid. In one embodiment, the rod can be cylindrical. In one embodiment, the rod can be longer than the width of the cutout 1100. The rod can be straight or curved. The rod 404 can be inserted into the holes 1101 on either side of the cutout 1100 to affix the spoke 400 to the distal opening of the outer tube 101. In one embodiment, the spoke 400 can be secured to the outer tube 101 and cannot be removed unless a lateral force is applied to the spoke to remove the rod from the holes in the cutout.

In one embodiment, the rod 404 can be an axis around which the spoke 400 can rotate from the retracted position to the expanded position. In one embodiment, the range of rotation of the spoke 400 can be approximately 90. In another embodiment, the range of rotation of the spoke can be between approximately 90° and approximately 180°. In one embodiment, the maximum range of rotation can ensure that the spokes 400 do not point upwards, e.g., towards the flange, as this configuration would irritate the inside of the gastric wall and/or cause pain to the patient. In one embodiment, the range of rotation of the spoke can be constrained by the upper wall of the cutout 1100. In one embodiment, each spoke 400 can include a tab 401a at the proximal end of the spoke. The tab 401a can extend inwards towards the center of the distal opening in the outer tube when the spoke 400 is in a retracted position. In one embodiment, the tab 401a can be attached to the rod 404.

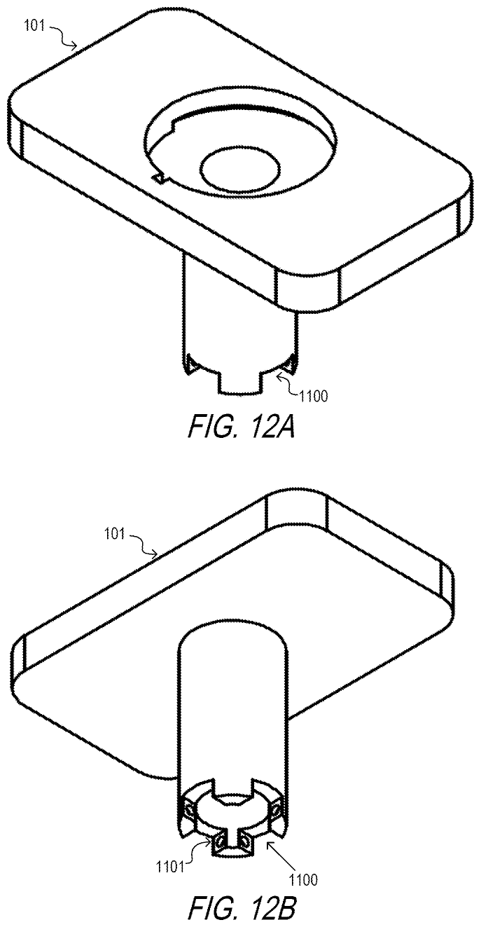

FIG. 12A illustrates an example embodiment of an outer tube 101 with cutouts 1100 for attachment of spokes. The cutouts can follow the curvature of the body of the outer tube. FIG. 12B is a bottom view of an example embodiment of an outer tube 101 with cutouts 1100 for attachment of spokes. There are holes 1101 in the wall of the outer tube 101 at each end of a cutout 1100. In one embodiment, the holes 1101 can be connected by a hollow channel in the wall of the outer tube 101. In another embodiment, the channels can terminate within the wall of the outer tube. According to an example embodiment, the outer body 101 can include three cutouts 1100 for three spokes, as illustrated in FIG. 12B.

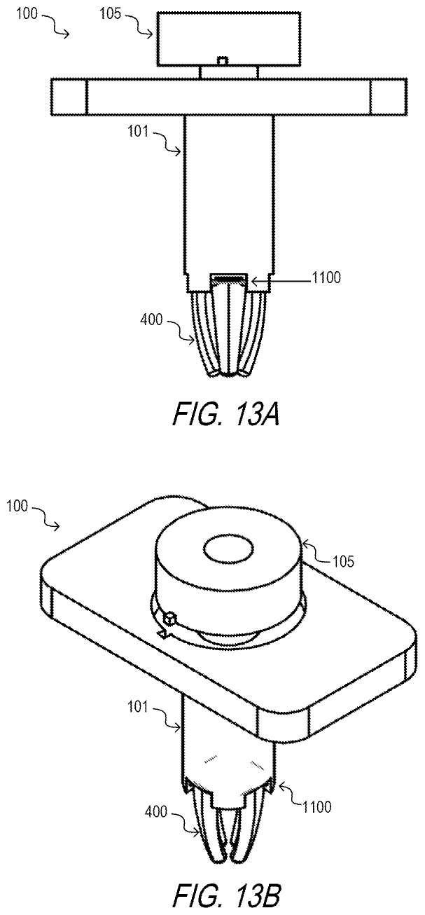

FIG. 13A illustrates an example embodiment of a gastrostomy tube 100 with hinged spokes 400 in a retracted position. The spokes 400 can point vertically in the retracted position. FIG. 13B is a top view of a gastrostomy tube 100 with hinged spokes 400 in a retracted position, according to one embodiment. The hinged spokes 400 can be maintained in a retracted position when the inner tube 105 is not fully inserted into the outer tube 101. The outer tube 101 can be inserted into the stomach when the hinged spokes 400 are in a retracted position.

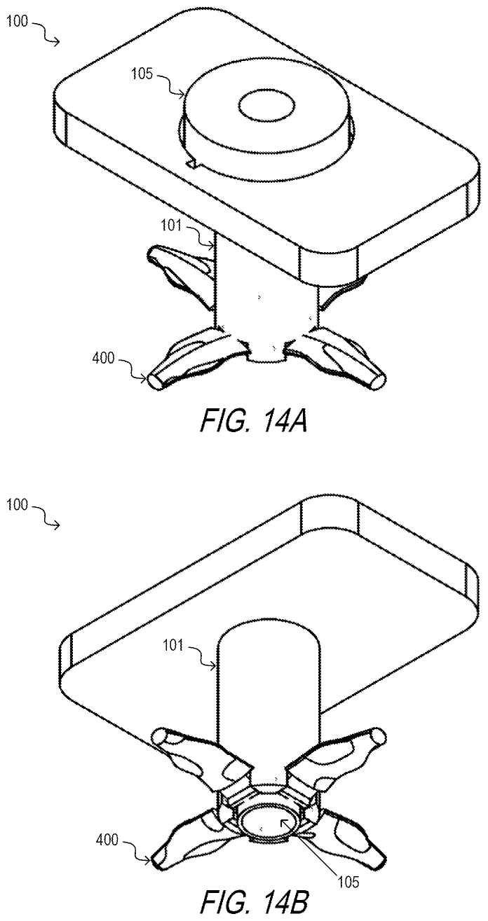

FIG. 14A illustrates an example embodiment of a gastrostomy tube 100 with hinged spokes 400 in an expanded position. The spokes 400 can be engaged in the expanded position by the inner tube 105. In one embodiment, the inner tube 105 can push down on the tab at the proximal end 401 of a hinged spoke 400. The downward force on the tab can cause the tab and the spoke 400 to rotate around the axis of the rod to the expanded position. In one embodiment, the rotation of the spoke 400 can be modulated by how much the inner tube 105 is inserted into the outer tube 101. In one embodiment, the inner tube 105 can be inserted until the spokes 400 meets the gastric wall. The gastrostomy tube can then be fixed in place by the spokes 400. In one embodiment, the distal end of the inner tube 105 can extend past the distal opening of the outer tube 101. The walls of the inner tube 105 can support the tabs to prevent the spokes from rotating back into the retracted position. FIG. 14B is an illustration of the distal end of a gastrostomy tube 100 with petal-shaped hinged spokes 400 in an expanded position, according to one embodiment. The inner tube 105 can be inserted into the outer tube 101, resulting in each of the hinged spokes 400 rotating around the axis of the rod to an expanded position. When the inner tube 105 is removed from the outer tube 101, the spokes 400 can rotate around the axis of the rod back to the retracted position. The outer tube 101 can then be removed from the stomach if desired.

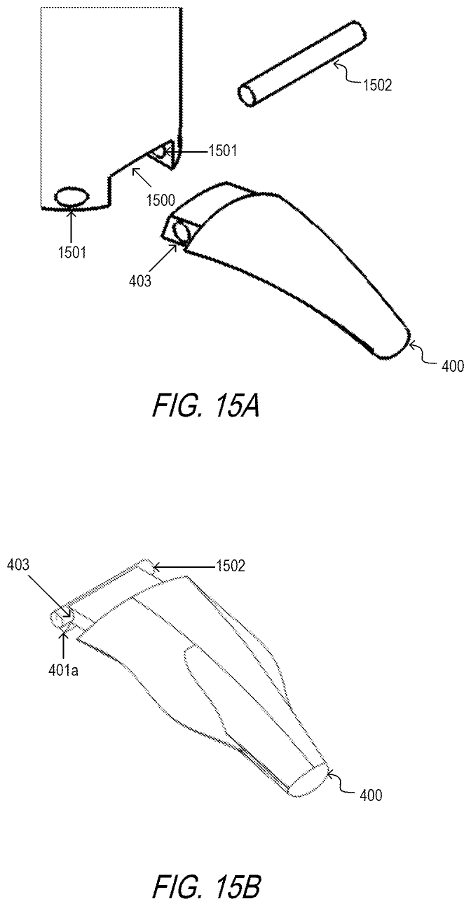

In one embodiment, the spokes can be attached to the outer body of the gastrostomy tube using an axle. FIG. 15A illustrates an example embodiment of a spoke 400 with a hollow channel 403 at the proximal end 401 of the spoke 400 for attaching to the gastrostomy tube. In one embodiment, the outer tube 101 of the gastrostomy tube can include a cutout 1500 in the wall of the outer tube. The cutout 1500 can include a pinhole 1501 in the outer wall of the outer tube on a first side of the cutout and a pinhole 1501 in the outer wall of the outer tube on an opposing side of the cutout. The spoke 400 can be inserted into the cutout 1500 such that the hollow channel 403 aligns with the pinholes 1501. An axle 1502 or pin can be inserted into a pinhole 1501 on a first side of the cutout. In one embodiment, the radius of the axle 1502 can be approximately equal to the radius of the pinholes 1501. The axle 1502 can continue through the hollow channel 403 in the proximal end of the spoke and through a pinhole 1501 in the opposing side of the cutout to the outer surface of the outer tube. In one embodiment, the axle 1502 can be rigid. The axle 1502 can secure the spoke 400 in the cutout and provide an axis of rotation for the spoke 400. FIG. 15B illustrates another example embodiment of a spoke 400 with a hollow channel 403 for an axle 1502. In one embodiment, the spoke 400 can be a petal-shaped spoke wherein the midsection is the same width as or wider than the proximal end 401 of the spoke 400. In one embodiment, the top surface of the spoke 400 can be composed of multiple planes. In one embodiment, the top surface of the spoke 400 can include a raised edge. In one embodiment, the hollow channel 403 can be partially open. For example, the channel can be a C-shaped channel. The partial opening in the hollow channel 403 can be smaller than the circumference of the axle 1502 so that the axle 1502 remains secure in the hollow channel at any angle of rotation. The axle 1502 can be inserted into the hollow channel 403 through holes on either end of the channel 403. In one embodiment, each spoke 400 can include a tab 401a at the proximal end of the spoke. The tab 401a can extend inwards towards the center of the distal opening in the outer tube when the spoke 400 is in a retracted position.

In one embodiment, the axle 1502 can be an axis around which the spoke 1502 can rotate from the retracted position to the expanded position. In one embodiment, the range of rotation of the spoke 400 can be approximately 90. In another embodiment, the range of rotation of the spoke can be between approximately 90° and approximately 180°. In one embodiment, the maximum range of rotation can ensure that the spokes 400 do not point upwards, e.g., towards the flange, as this configuration would irritate the inside of the gastric wall and/or cause pain to the patient. The range of rotation of the spoke can be constrained by the upper wall of the cutout 1100.

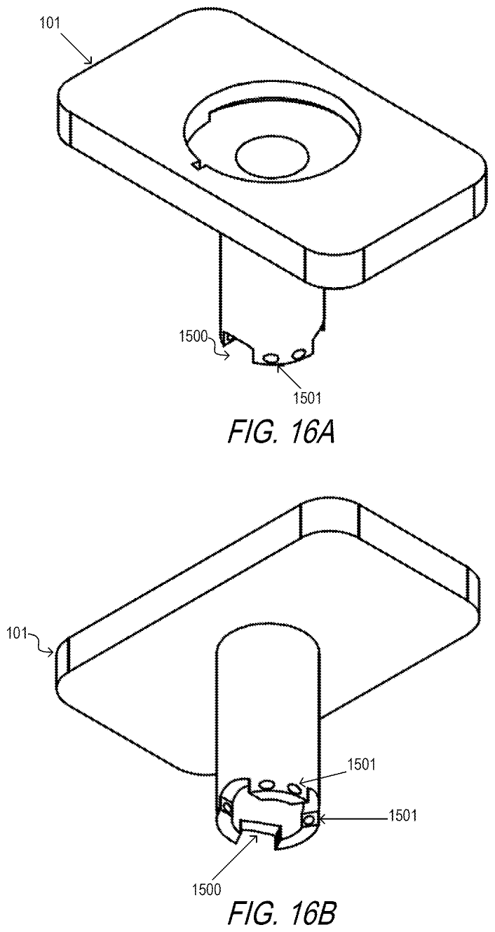

FIG. 16A is an example embodiment of an outer tube 101 with cutouts 1500 for spokes. Each cutout can include a first pinhole on a first wall of the cutout and a second pinhole on an opposing wall of the cutout for securing the spoke to the cutout. In a preferred embodiment, the first pinhole and the second pinhole can be the same size and can be directly opposite each other. In one embodiment, the cutout 1500 can be rectangular. In another embodiment, the cutout 1500 can be rounded. FIG. 16B is an example embodiment of an outer tube 101 with cutouts 1500 for spokes. The pinholes 1501 can lead from the cutout to the outer surface of the outer tube 101. The axles can be inserted into the cutouts from the pinholes at the outer surface of the outer tube 101.

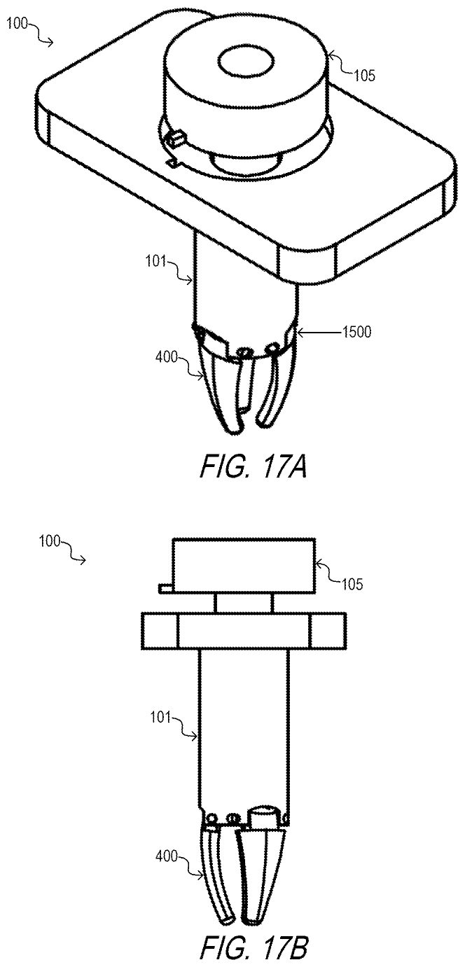

FIG. 17A is an example embodiment of a gastrostomy tube 100 with spokes 400 attached to the outer tube 101 using axles wherein the spokes 400 are in a retracted position. The spokes 400 can fit into the cutouts 1500 in the outer tube. In one embodiment, the outer tube 101 can have three cutouts for three spokes. In one embodiment, the tapered ends of the spokes can meet when the spokes are in a retracted position. FIG. 17B is a profile view of an example embodiment of a gastrostomy tube 100 with spokes 400 attached to the outer tube 101 using axles wherein the spokes 400 are in a retracted position.

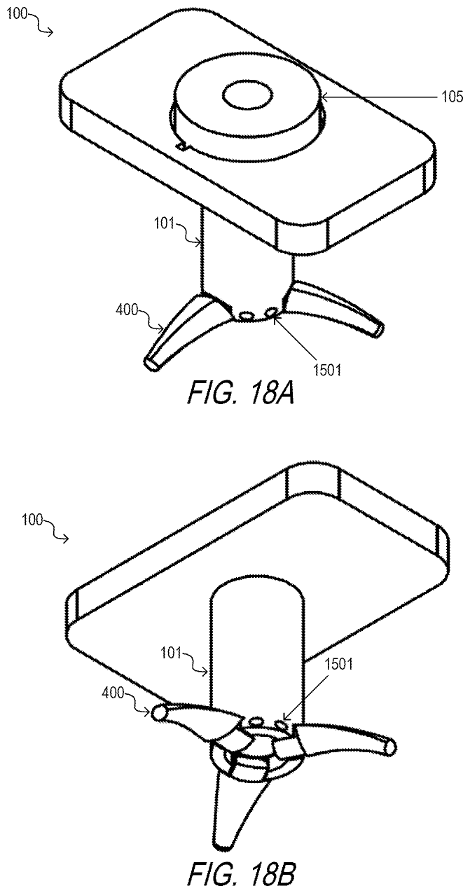

FIG. 18A is an example embodiment of a gastrostomy tube 100 with spokes 400 attached to the outer tube 101 using axles wherein the spokes 400 are in an expanded position. The spokes 400 can be engaged in the expanded position by the inner tube 105. In one embodiment, the inner tube 105 can push down on the tab at the proximal end of the spoke 400. The downward force on the tab can cause the tab and the spoke 400 to rotate around the axle to the expanded position. In one embodiment, the rotation of the spoke 400 can be modulated by how much the inner tube 105 is inserted into the outer tube 101. In one embodiment, the inner tube 105 can be inserted until the spokes 400 meets the gastric wall. The gastrostomy tube can then be fixed in place by the spokes 400. In one embodiment, the distal end of the inner tube 105 can extend past the distal opening of the outer tube 101. The walls of the inner tube 105 can support the tabs to prevent the spokes from rotating back into the retracted position. FIG. 18B is an illustration of the distal end of a gastrostomy tube 100 with spokes 400 attached to the outer tube 101 using axles according to one embodiment. The spokes 400 are in an expanded position. In one embodiment, the shape and/or surface of the spokes can provide more friction between the spoke and the inside of the gastric wall in order to secure the gastrostomy tube inside the stomach.

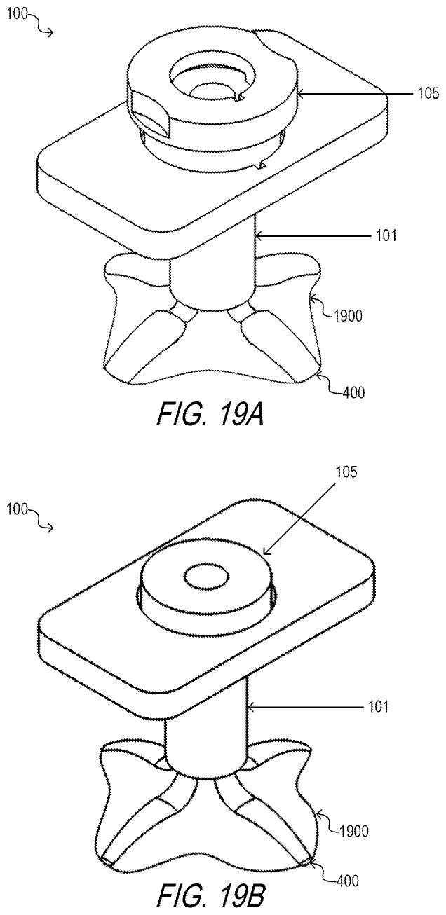

In one embodiment, the gastrostomy tube can include a membrane between the spokes, as illustrated in FIG. 19A. The membrane 1900 can form a continuous surface surrounding the spokes 400 to prevent tissue growth in the gaps between spokes when the spokes are in an expanded position. The continuous surface can be a circle. In another embodiment, the continuous surface can be an irregular shape. In one embodiment, the membrane can be composed of a biocompatible material. The membrane can be smooth or can include textured regions. In one embodiment, the membrane can cover the upper surface or the bottom surface of the spokes. In another embodiment, the membrane can be attached between the spokes. In one embodiment, the membrane can extend past the length of the spokes. A longer membrane can provide a larger barrier to prevent tissue growth near the spokes. In another embodiment, the membrane can extend up to any point along the length of the spokes, including the full length of the spokes. In one embodiment, the membrane 1900 can be attached to the distal opening of the outer tube 101. In one embodiment, the membrane 1900 can be attached to the inner tube 105. FIG. 19B illustrates an example embodiment of flexible spokes 400 attached to the inner tube 105 of the gastrostomy tube with a membrane 1900 between the spokes. The membrane can fold when the spokes are in the retracted position in order to maintain the streamlined shape of the retracted spokes for insertion into the stomach.

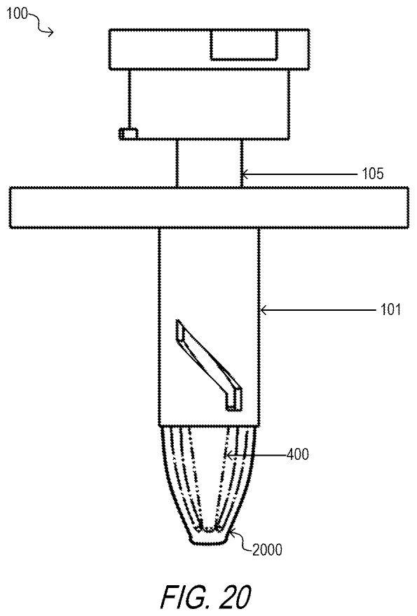

In one embodiment, the spokes can be surrounded by a biodegradable sheath in the retracted position. An exemplary embodiment of a biodegradable sheath is illustrated in FIG. 20. The biodegradable sheath 2000 can be placed over the spokes 400. In one embodiment, the biodegradable sheath 2000 can be a cap wherein the cap fits onto the spokes 400 when the spokes are retracted. The biodegradable sheath 2000 can be placed over spokes attached to the outer tube 101 or the inner tube 105. In one embodiment, the biodegradable sheath 2000 can tear open when the spokes are engaged in an expanded position. For example, the biodegradable sheath 2000 can be perforated to be torn open easily. In another embodiment, the biodegradable sheath 2000 can degrade or dissolve in the body before the spokes are engaged in the expanded position.

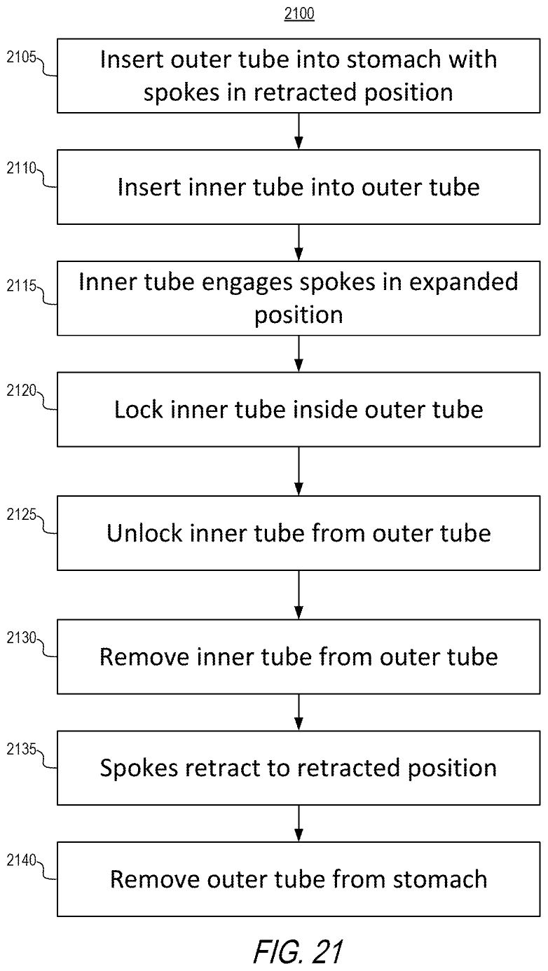

FIG. 21 illustrates a method 2100 for using the gastrostomy tube according to an embodiment. The outer tube can be inserted into an incision in the abdomen and stomach in step 2105. The spokes are in a retracted position when the outer tube is inserted into the stomach. The inner tube can be inserted into the outer tube in step 2110. The distal end of the inner tube can engage the spokes in an expanded position in step 2115. In one embodiment, the distal end of the inner tube can exert a force on the spokes to engage the spokes in the expanded position. The inner tube can be locked into place in the outer tube in step 2120, e.g., by rotating the inner tube. The outer and inner tube can be used to feed the patient. In one embodiment, an extension tube can be inserted into the inner tube and locked into place for feeding the patient. When the patient is ready for the gastrostomy tube to be removed, the inner tube can be unlocked from the outer tube in step 2125. The inner tube can be removed from the outer tube in step 2130. The removal of the inner tube can result in the spokes returning to a retracted position in step 2135. The outer tube can then be removed from the incision when the spokes are in a retracted position in step 2140.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A gastrostomy tube for insertion through an abdomen and into a stomach, comprising:
a cylindrical outer tube including a first proximal opening and a first distal opening;
a flange surrounding the first proximal opening of the outer tube;
a detachable cylindrical inner tube including a second proximal opening and a second distal opening; and
one or more spokes attached to a distal end of the gastrostomy tube,
wherein the one or more spokes include an actuatable portion configured to rotate the one or more spokes between a retracted position and an expanded position,
wherein the inner tube is insertable into the first proximal opening of the outer tube,
wherein the one or more spokes are rotatable into the expanded position when the inner tube is inserted through the first distal opening of the outer tube, and
wherein the one or more spokes are substantially perpendicular to the outer tube in the expanded position.

2. The gastrostomy tube of claim 1, wherein the flange of the outer tube includes a locking mechanism configured to secure the inner tube inside the outer tube.

3. The gastrostomy tube of claim 1, wherein the outer tube includes a locking mechanism along a body of the outer tube configured to secure the inner tube inside the outer tube.

4. The gastrostomy tube of claim 1, wherein the one or more spokes are attached to the first distal opening of the outer tube.

5. The gastrostomy tube of claim 4, wherein the first distal opening of the outer tube includes one or more cutouts for attachment of the one or more spokes.

6. The gastrostomy tube of claim 1, wherein the actuatable portion includes a flexible joint.

7. The gastrostomy tube of claim 6, wherein the flexible joint is configured for a continuous range of motion.

8. The gastrostomy tube of claim 6, wherein a position of the one or more spokes corresponds to a curvature of the flexible joint.

9. The gastrostomy tube of claim 1, wherein the actuatable portion includes an axle.

10. The gastrostomy tube of claim 1, wherein the one or more spokes are attached to the second distal opening of the inner tube.

11. The gastrostomy tube of claim 10, wherein the actuatable portion is a flexible region of the one or more spokes.

12. The gastrostomy tube of claim 11, wherein a position of the one or more spokes corresponds to a curvature of the flexible region.

13. The gastrostomy tube of claim 1, further comprising a membrane extending from the one or more spokes.

14. The gastrostomy tube of claim 1, further comprising a biodegradable sheath configured to fit over the one or more spokes in the retracted position.

15. A method for inserting a gastrostomy tube through an abdomen and into a stomach, the method comprising:
arranging one or more spokes attached to a distal opening of the gastrostomy tube in a retracted position;
inserting an outer tube of the gastrostomy tube through the abdomen and into the stomach;
inserting an inner tube into the outer tube;
engaging, with the inner tube, an actuatable portion of the one or more spokes to rotate the one or more spokes into an expanded position, the one or more spokes being substantially perpendicular to the outer tube in the expanded position; and
locking the inner tube inside the outer tube.

16. The method of claim 15, wherein the one or more spokes is attached to a distal opening of the outer tube.

17. The method of claim 15, wherein the one or more spokes is attached to a distal opening of the inner tube.

18. The method of claim 15, further comprising the actuatable portion including a flexible joint.

19. The method of claim 18, the flexible joint being configured for a continuous range of motion.

20. The method of claim 15, further comprising inserting an extension tube into the inner tube.
